(19) United States
(12) Patent Application Publication
(10) Pub. No.: US 2026/0272691 A1
(43) Pub. Date: Sep. 17, 2026

(54) Systems And Methods For Suppressing Essential Tremors

(71) Applicant: Hutchinson Technology Incorporated, Hutchinson, MN (US)

(72) Inventor: Mark A. Miller, Hutchinson, MN (US)

(21) Appl. No.: 19/564,071

(22) Filed: Mar. 11, 2026

Related U.S. Application Data
(60) Provisional application No. 63/772,238, filed on Mar. 14, 2025.

(51) Int. Cl. A61F 5/01; A61N 1/04

(52) U.S. Cl. CPC A61F 5/0118; A61N 1/0452

(57) ABSTRACT

The present embodiments relate to systems and methods for suppressing essential tremors using a shape memory alloy (SMA) devices. The systems as described herein can be designed to contract/relax muscles in response to a tremor and also allow for movement during normal motion of the user. The designs can implement SMA actuators having a straight SMA wire concept or a metal cross springs in a stacked “X” configuration (also referred to as Xmorph) concept that generates enough force to hold and move the weight of the hand and/or wrist of the user while also generating sufficient force to suppress tremors of the user.

### CROSS REFERENCE TO RELATED APPLICATIONS

The present application claims priority to United States Provisional Patent Application No. 63/772,238, titled “SYSTEMS AND METHODS FOR SUPPRESSING ESSENTIAL TREMORS,” and filed Mar. 14, 2025, the entirety of which is incorporated by reference herein.

### TECHNICAL FIELD

Embodiments of the invention relate to the field of shape memory alloy systems. More particularly, embodiments of the invention relate to systems to suppress essential tremors using shape memory alloy actuators.

### BACKGROUND

Essential Tremors are involuntary movements that commonly affect parts of the body such as the hands of a patient. Tremors can comprise involuntary or rhythmic movements that can range from mild to severe.

Various devices can be implemented in an attempt to mitigate essential tremors. Some example devices can be rigid in an attempt to prevent the patient's hands from moving during a tremor. However, many such devices are burdensome and do not allow for much movement when tremors are not present. Other devices may be more flexible, providing more comfort to the patient during use, but may not provide much support during tremors.

### SUMMARY

The present embodiments relate to systems and methods for suppressing essential tremors using a shape memory alloy (SMA) devices. The systems and methods as described herein can be designed to contract/relax muscles in response to a tremor and also allow for movement during normal motion of the user. The designs can implement SMA actuators having a straight SMA wire concept, or a stacked concept with cross or “X” configuration of metal cross springs (also referred to herein as “Xmorph”), that can generate enough force to hold and move the weight of the hand and/or wrist of the user while also generating sufficient force to suppress tremors of the user.

In a first example embodiment, a system for suppressing tremors in a user is provided. The system can include a first component configured to connect to the user. The system can also include a second component configured to connect to the user. The system can also include a set of shape memory alloy (SMA) wires connecting at a first end to the first component and at a second end to the second component. Actuation of the set of SMA wires can be configured to contract/relax muscles of a user in response to a tremor and allow for movement during normal motion of the user.

In some instances, the first component is configured to surround a wrist of the user, and wherein the second component is configured to surround a forearm of the user.

In some instances, the first component and the second component comprises a strap with a fastener to secure the strap to the user.

In some instances, the set of SMA wires further comprises a first pair of SMA wires on a first side of the first component and the second component and a second pair of SMA wires on a second side of the first component and the second component opposite to the first side.

In some instances, the system can also include a set of connection pads on each of the first component and the second component, wherein each of the set of connection pads provides a flat surface to engage the first end or the second end of any of the set of SMA wires to the first component or the second component.

In some instances, the first component comprises a set of ball bearing sliding surfaces to allow for sliding movement of the connection pads on the first component.

In some instances, the system can also include a glove component configured to engage with a hand and/or fingers of the user.

In some instances, the system can also include a set of metal wires connecting the glove component to the first component.

In some instances, the system can also include a series of links connecting the first component to the second component and disposed adjacent to any of the set of SMA wires.

In some instances, the system can also include a silicon sleeve disposed around any of the set of SMA wires.

In some instances, the system can also include a set of cross-members disposed between the first component and the second component, wherein the set of cross-members cross at a central point.

In some instances, the system can also include a stack of actuators disposed between the first component and the second component, wherein each actuator in the stack of actuators comprises the set of cross-members and the set of SMA wires.

In some instances, the stack of actuators comprises at least ten actuators in a stacked configuration.

In some instances, the system can also include one or more Electric Muscle Simulation (EMS) devices configured to be disposed on the user, wherein each of the one or more EMS devices comprises a SMA element configured to actuate and cause activation of a muscle of the user.

In some instances, wherein the one or more SMA actuators are configured to generate sufficient force to hold and move the weight of the users'hand and/or wrist while also generating sufficient force to suppress tremors in the user.

In another example, a device is provided. The device can include a glove component configured to connect to a hand and/or fingers of a user. The device can also include a wrist component configured to connect to a wrist region of the user. The device can also include a forearm component configured to connect to a forearm region of the user. The device can also include a set of metal wires disposed between the glove component and the wrist component. The device can also include a set of shape memory alloy (SMA) wires connecting at a first end to the wrist component and at a second end to the forearm component. The set of SMA wires can further include a first pair of SMA wires on a first side of the first component and the second component and a second pair of SMA wires on a second side of the first component and the second component opposite to the first side.

In some instances, the device can also include a set of connection pads on each of the first component and the second component, wherein each of the set of connection pads provides a flat surface to engage the first end or the second end of any of the set of SMA wires to the first component or the second component.

In some instances, the device can also include a series of links connecting the first component to the second component and disposed adjacent to any of the set of SMA wires.

In some instances, the device can also include a set of cross-members disposed between the first component and the second component, wherein the set of cross-members cross at a central point.

In some instances, the device can also include one or more Electric Muscle Simulation (EMS) devices configured to be disposed on the user, wherein each of the one or more EMS devices comprises a SMA element configured to actuate and cause activation of a muscle of the user.

### BRIEF DESCRIPTION OF THE DRAWINGS

Embodiments of the present invention are illustrated by way of example and not limitation in the figures of the accompanying drawings, in which like references indicate similar elements and in which:

FIG. 1 is an example SMA straight wire essential tremor (ET) actuator design, according to some embodiments.

FIGS. 2A-B illustrate views of example Xmorph configurations, according to some embodiments.

FIG. 3 illustrates an example Electric Muscle Simulation (EMS) system, according to some embodiments.

FIGS. 4A-B illustrate views of an example SMA straight wire design, according to some embodiments.

FIG. 5 illustrates an example SMA straight wire design that can hold the weight of the hand, according to some embodiments.

FIGS. 6A-B illustrate views example SMA straight wire design, according to some embodiments.

FIGS. 7A-7B illustrate views of a SMA straight wire design illustrating example reaction forces, according to some embodiments.

FIG. 8 illustrates a SMA straight wire design with a semi-compliant link, according to some embodiments.

FIGS. 9A-9B illustrate graphical representations of parameters relating to a SMA straight wire design with an example response time, according to some embodiments.

FIG. 10 is an example SMA Xmorph design, according to some embodiments.

FIG. 11 illustrates another example SMA Xmorph design, according to some embodiments.

FIGS. 12A-C illustrate views an example EMS system for essential tremor control, according to some embodiments.

FIG. 13 is an example networked computing arrangement which may be used in the systems and methods described herein.

FIG. 14 shows an example computing device which may be used in the systems and methods described herein.

### DETAILED DESCRIPTION

Essential Tremors are involuntary movements that commonly affect parts of the body such as the hands of a patient. Tremors can comprise involuntary or rhythmic movements that can range from mild to severe.

Various devices can be implemented in an attempt to mitigate essential tremors. Some example devices can be rigid in an attempt to prevent the patient's hands from moving during a tremor. However, many such devices are burdensome and do not allow for much movement when tremors are not present. Other devices may be more flexible, providing more comfort to the patient during use, but may not provide sufficient support during tremors.

The present embodiments relate to systems and methods for suppressing essential tremors using a shape memory alloy (SMA) devices. The systems and methods as described herein can be designed to contract/relax muscles in response to a tremor and also allow for movement during normal motion of the user. The designs can implement SMA actuators having a straight SMA wire concept, or a stacked concept with cross or “X” configuration of metal cross springs (also referred to herein as “Xmorph”), that can generate enough force to hold and move the weight of the hand and/or wrist of the user while also generating sufficient force to suppress tremors of the user.

As an operating principle, an internal measurement unit (IMU) can be placed on the hand of the user. Actuators can be mounted to the forearm and coupled to the wrist via an inner wire moving within a hollow, stiffer outer casing, sometimes referred to as Bowden cables. When tremor motion is detected by the IMU, actuators can contract/relax the cables to reduce unwanted motion.

A practical example using these systems and methods as described herein can include any of an arm mount, a battery, a single servo, a hand brace, and an IMU or other electronics. In some instances, the device can include a battery, a glove, a wrist cuff, Bowden Cables, two servos, and an IMU and other electronics. It should be noted that the examples of the system used on a human hand, wrist, and arm, are not intended to be limiting. Various other body parts could be equipped with the same or similar structures as described herein, to limit trembling.

The designs as described herein can provide for tremor reduction that can target 80% or more tremor suppression, while allowing for voluntary movement and suppressing involuntary tremors. The designs can optimize for cost and lower power consumption in mind, with a minimal weight for user comfort and optimized experience.

The designs can have various design criteria. For example, a maximum estimated torque on any one particular cable can be around 0.5 Nm, and an estimated mass of the hand can be around 0.5 kg. A maximum displacement amplitude to be controlled can be ~1 inch, and a tremor frequency to be controlled can be around 6 Hz. An example use case can be to stabilize the tremor while allowing for natural hand movements. An actuation frequency range can be around 100 to 200 Hz. The actuator can provide state feedback that can include including position, speed, torque, and temperature, similar to servo encoders. The actuator can be compact, enabling the construction of a wearable glove that remains at room temperature during operation.

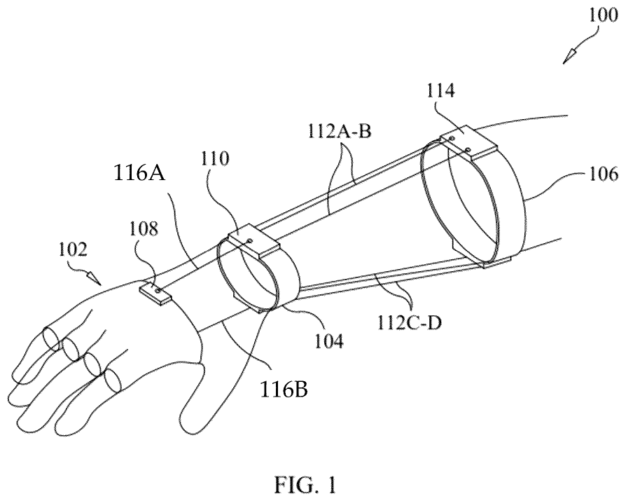

FIG. 1 is an example SMA straight wire essential tremor (ET) actuator design 100. As shown in FIG. 1, the design 100 can include a glove 102 configured to go over the hand and partially over fingers of the user at a first end, a wrist support 104, and a forearm support 106. A pair of metal (e.g., steel) links 116A-B can be between the glove 102 and wrist support 104. Each link 116A-B can connect at a first end 108 to the glove 102 and a second end to a connection portion 110 of the steel link. The connection portion 110 can connect the wrist support 104 to the steel links 116A-B and SMA wires 112A-D. The connection portion 110 can further provide an electrical current to SMA wires 112A-D and/or isolate the wires 112A-D into separate circuits.

The SMA wires 112A-D can connect at a first end to the connection portion(s) 110 of the wrist support 104 and at a second end to the connection portion(s) 114 of the forearm support. The steel links 116A-B can provide support to the hand and wrist of the user, while SMA wires 112A-D can actuate in response to a tremor being detected (e.g., via an IMU unit (not shown) to mitigate arm movements caused by the tremor.

In some examples, the design can include a SMA straight wire essential tremor (ET) actuator. This design can have a higher force/torque and can be light weight and low profile. The design can have a high force on upper forearm that could restrict blood flow. Further, additional design and component complexity can reduce force. The design can also implement active cooling.

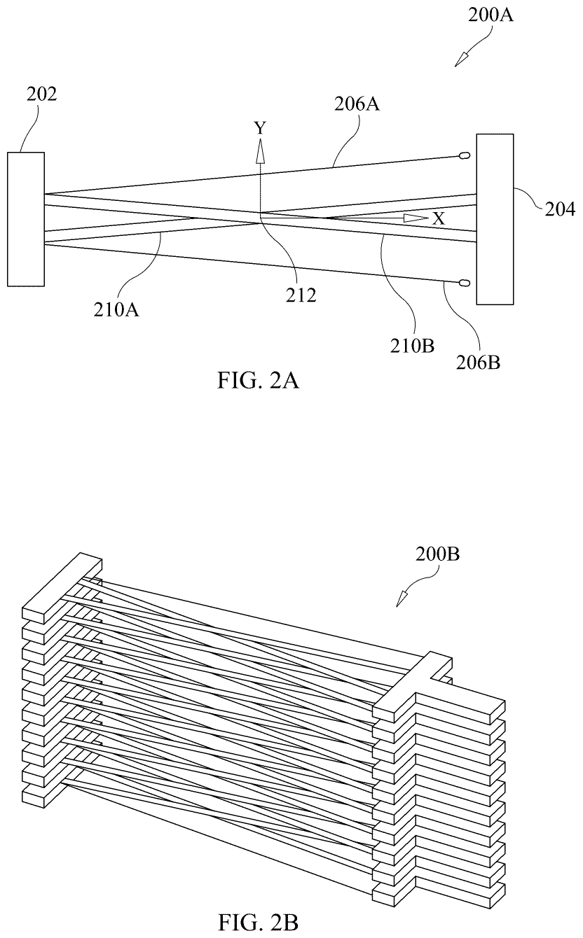

FIGS. 2A-B illustrate views of example Xmorph configurations 200A-B. The term Xmorph is used as a general description of the devices with metal cross springs arranged in a cross or “X” type configuration.

As shown in FIG. 2A, a single Xmorph device is provided. As shown in FIG. 2A, the Xmorph device 200A can include ends 202, 204 with metal supports 210A-B extending between ends 202, 204. The metal supports 210A-B can be in a cross-configuration, with a central point 212 identifying where the supports 210A-B cross. Further, SMA wires 206A-B extend between ends 202, 204. The SMA wires 206A-B can be on opposing sides of the metal supports 210A-B.

Actuation of the SMA wires 206A-B can cause the device 200A to actuate in a positive or negative direction relative to a user when positioned near or on the user. For example, in response to a tremor occurring in an arm or hand of the user, the SMA wires 206A-B as described herein can actuate by contracting or expanding one or more muscles or tendons in the arm of the user, causing the muscles or tendons to activate and thereby lessen the impact of the tremor.

FIG. 2B illustrates a stack of Xmorph devices. Any number of Xmorph devices can be stacked to provide additional support and torque to suppress tremors as described herein.

Another design can include stacked Xmorph motors on the forearm with Bowden cables. This design can include a light weight device. This design can use around 20 stacked Xmorphs to generate needed torque.

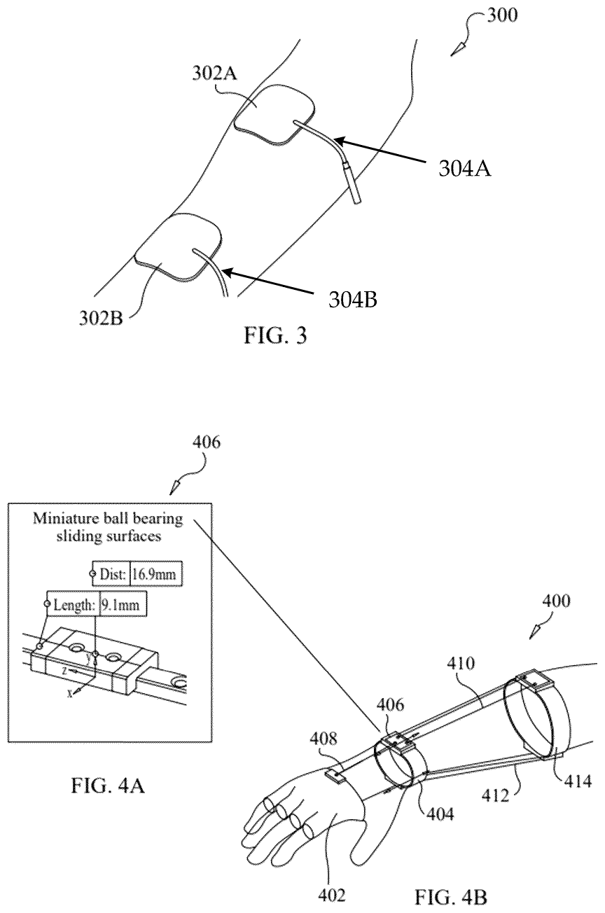

FIG. 3 illustrates an example Electric Muscle Simulation (EMS) system 300. As shown in FIG. 3, the EMS system 300 can include a number of EMS actuators 302A-B disposed on an arm of the user. EMS actuators 302A-B can include pads that adhere to the skin of the user, while SMA elements can actuate at the pads to activate muscles. The pads 302A-B as described herein can have an end of the SMA wire 304A-B attached to the pad 302A-B. The end of the SMA wire 304A-B upon actuation can cause the pad 302A-B to expand or contract and thereby cause underlying muscles or tendons to relax or activate to alleviate conditions such as tremors.

In some instances, one or more SMA wires 304A-B can be disposed within the pads 302A-B. The SMA wires 304A-B can be disposed within distinct layers of the pad 302A-B, separated from the user by a layer configured to contact the skin of the user. The SMA wires 304A-B can form a pattern within the pad 302A-B, such as a web-like pattern, for instance. Upon actuation of the SMA wires 304A-B, part or all of the pad 302A-B can contract or expand on the arm of the user, applying a force into the arm of the user to mitigate the impact of a tremor or similar movement.

This design can use opposing muscles to suppress motion without any additional actuators. This design can have high force and frequency potential, that is small and light weight. This design can have a control capability to smoothly activate muscles. Further, four or more pads can be adhered to the skin for use.

In some embodiments, the systems can implement a SMA straight wire design concept. FIGS. 4A-B illustrate views of an example SMA straight wire design. As shown in FIG. 4B, the design 400 can include a glove 402, wrist strap 404, and forearm strap 414. The glove 402 and wrist strap 404 can connect via steel links 408. Further, a sliding surface 406 on the wrist strap 404 can allow for sliding movement of the wrist strap 404, links 408, and/or SMA wires 410, 412. The SMA wires 410, 412 can be disposed between wrist strap 404 and forearm strap 414. The design as shown in FIG. 4B can include four SMA Wires that are around ~180 mm long. The design can also include two steel links, a wrist strap with two sliding surfaces, a forearm strap anchor, and an actuated glove.

FIG. 4A illustrates a detailed view of the sliding surfaces 406. The sliding surfaces 406 can include miniature ball bearing sliding surfaces with a distance of around 16.9 mm and a length of around 9.1 mm.

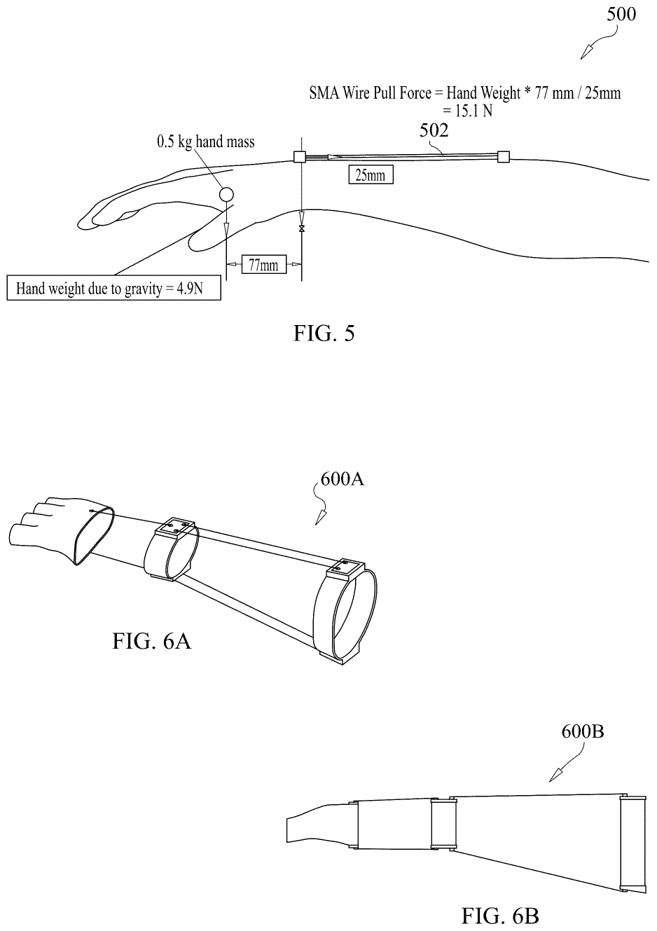

In some embodiments, the SMA straight wire design can hold the weight of the hand. FIG. 5 illustrates an example SMA straight wire design 500 that can hold the weight of the hand. In FIG. 5, the hand mass can be around 0.5 kg, a hand weight due to gravity can be around 4.9 N, a first distance can be around 77 mm and a first height can be around 25 mm.

In the design as described in FIG. 5, SMA wires 502 can be able to generate enough force to hold and move the weight of the hand. A minimum 15 N of pull force can be needed to just hold hand static (0.38 Nm torque). This design can use two SMA wires that are around 0.2-0.3 mm in diameter to generate this force.

FIGS. 6A-6B illustrate views 600A-B example SMA straight wire design. In the design as illustrated in FIGS. 6A-6B, hand rotation can be around ±9° (~±1 inch) with gravity and 0.5 kg hand mass. Hand mass/gravity can impact power required by SMA wires. The design as described herein can provide ~45% more power to top wires to overcome the weight of a user's hand.

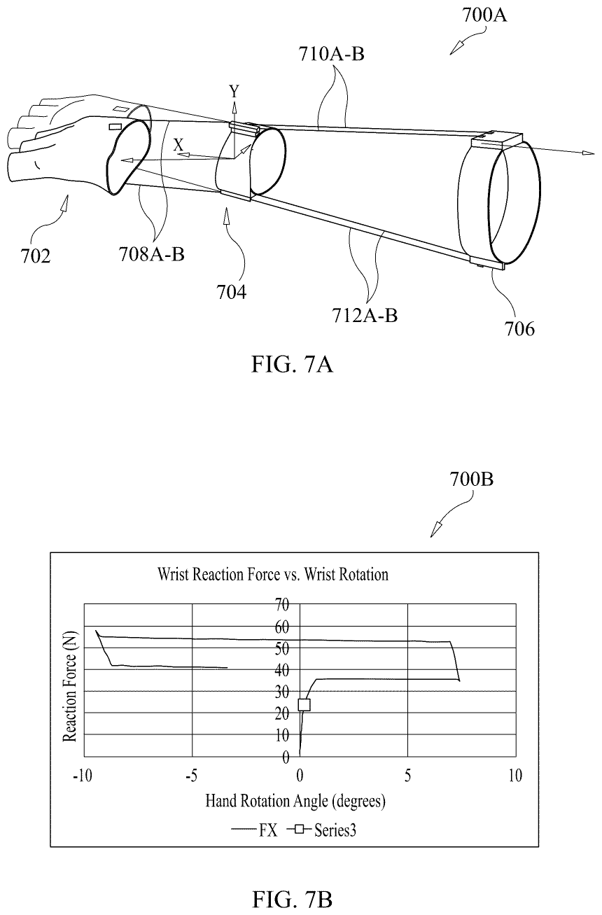

FIGS. 7A-7B illustrate views 700A-B of a SMA straight wire design illustrating example wrist reaction force against wrist rotation of a user. In the design of FIGS. 7A-7B, there can be a large amount of reaction force for this design that can be around 40 N (9 lbf). This force can pull the straps toward the hand causing loss of actuation stroke. Alternatively, the force may be so high that blood flow could be impacted. Assuming a human skin coefficient of friction is around 0.8, a total force required can be equal to a normal force of around 40 N/0.8 or 50N. The designs can have an improved way to anchor SMA wires to forearm than strap.

Further, in FIG. 7A, device 700A can include a glove component 702, a wrist component 704, and a forearm component 706. Sets of SMA wires 710A-B and 712A-B can be disposed between wrist component 704 and forearm component 706. Steel wires 708A-B can be between the glove component 702 and wrist component 704.

FIG. 7B is a graphical representation of an example wrist reaction force and a wrist rotation. More particularly, in FIG. 7B, the graph 700B can represent an example wrist reaction force against wrist rotation of a user, comparing a hand rotation angle against a reaction force. This can illustrate requisite specifications for the systems as described herein to be able to withstand repeated rotation and forces applied by the user during use of these systems.

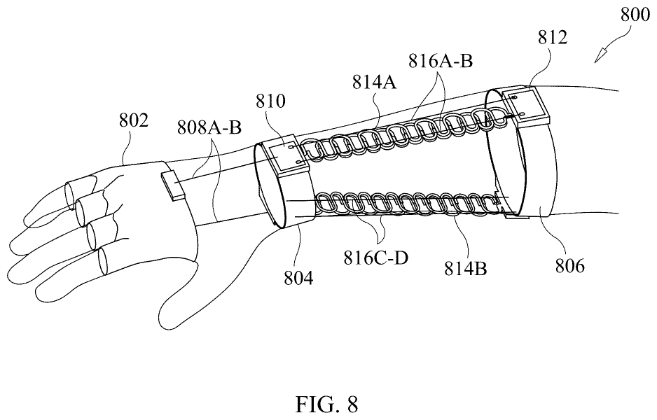

FIG. 8 illustrates a SMA straight wire design 800 with a semi-compliant link. As shown in FIG. 8, instead of only straight wires as shown above, a set of steel loops or links 808A-B can connect glove 802 and wrist strap 804. Further, a set of SMA wires 816A-D can be disposed between wrist strap 804 and forearm strap 806. A semi-compliant link 814A-B can be disposed between wrist strap 804 and forearm strap 806 adjacent to SMA wires 816A-B and 816C-D. Any of the SMA wires 816A-D and semi-compliant links 814A-B can connect to connection pad 810 at the wrist strap 804 and pad 812 at the forearm strap 806.

In the design of FIG. 8, to help distribute the forces applied by the arm of the user, such as a clamping force (e.g., a compressive force generated by muscles around joints of the arm), semi-compliant links can be attached to the wrist and forearm sections. This all can be sewn into a tight sleeve and additional straps can be used. The semi-compliant link can be designed to be axially rigid and torsionally compliant to allow natural wrist motion in any direction and help separate SMA wire from the arm. This can also help distribute forces applied by the user but still have a relatively large force requirement. However, a SMA actuator “sleeve” may slide down toward the hand in use to reduce the force required in this design.

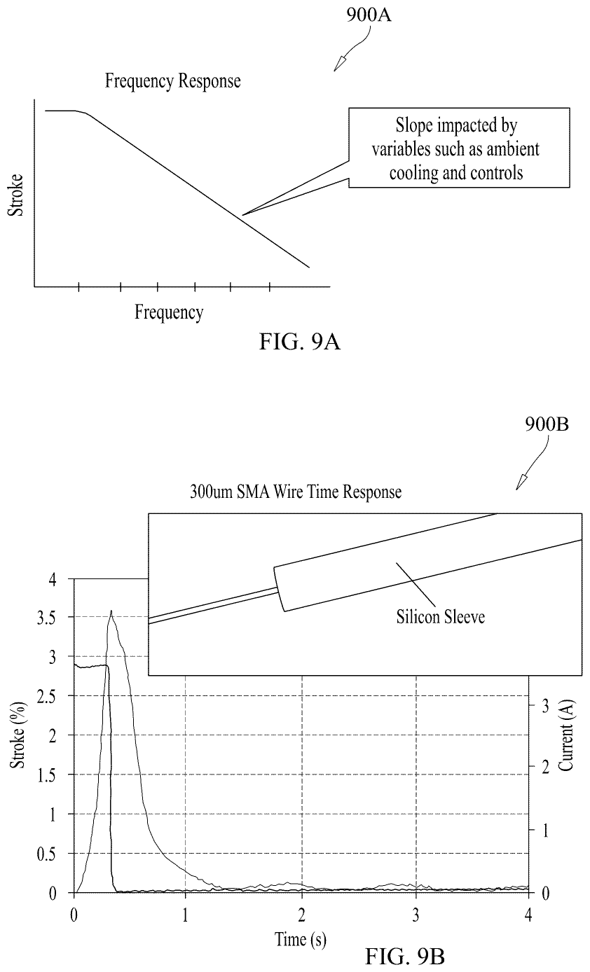

FIGS. 9A-9B illustrate graphical representations 900A-B of parameters relating to a SMA straight wire design with an example response time. For instance, FIG. 9A depicts a graphical representation 900A of a frequency response comparing a frequency and stroke of the designs as described herein. The slope can be impacted by variables such as ambient cooling and controls of the device. FIG. 9B illustrates a wire response time comparing time and stroke of the SMA wire in the straight wire design.

In the designs as described herein, adding a silicon sleeve around the SMA wire may be able to enhance cooling response. The design can provide a full±stroke response at 1 Hz, and the stroke can degrade at frequencies >1 Hz. Active cooling can improve response time by another 4×.

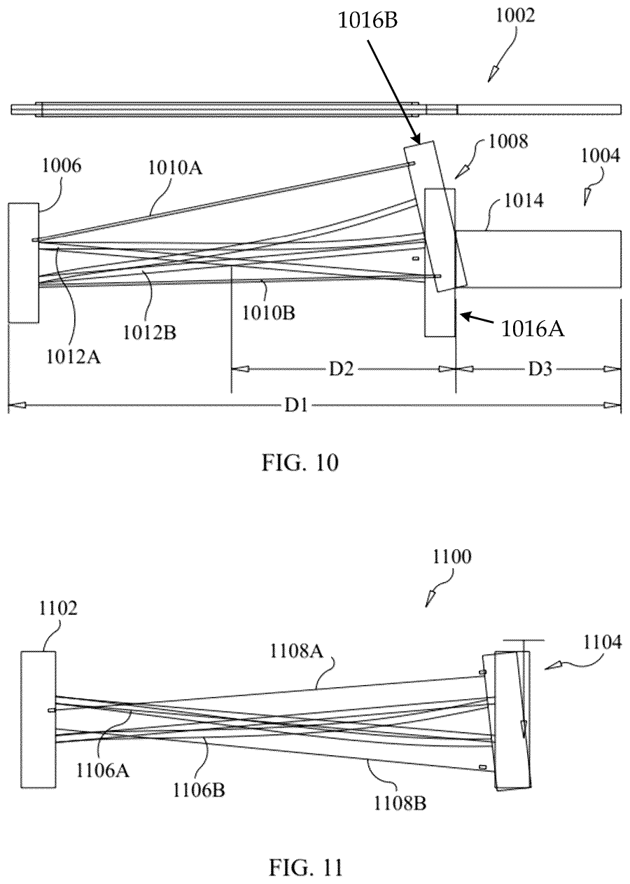

FIG. 10 is an example SMA Xmorph design. As shown in FIG. 10, the design can be viewed from a first, sid-on view 1002 depicting a thickness of the SMA Xmorph design and a second, top down view 1004 depicting the SMA Xmorph design. The SMA Xmorph design can include a first end 1006 that is configured to be fixed and a second end 1008 configured to move in response to actuation of SMA wires 1010A-B. The second end 1008 can be connected to a servo motor 1014 and can move from an original position 1016A to an actuated position 1016B. Further, cross-members 1012A-B can be disposed between first end 1006 and second end 1008 and can cross one another.

The design 1004 can have a first distance D1 along an entirety of the SMA Xmorph design that can be around 30 mm. A second distance D2 can extend from a center of the SMA Xmorph design to the second end 1008 can be around 15 nm. Further, a third distance D3 extending from the second end 1008 to an end of a servo motor 1014.

In FIG. 10, servo motors (e.g., 1014) can be replaced with SMA Xmorph Stack “motor” to drive Bowden cables (e.g., allow the cables to constrict or expand). The design may need >±0.5 mm of stroke to drive hand up and down ±10°. The stroke at the end can be around ±3.6 mm. Therefore, a ≥6 mm tip can be used to attach Bowden cable to, to generate >0.5 mm of stroke.

FIG. 11 is an example SMA Xmorph design. In FIG. 11, a total actuator torque can be around 500 Nmm, and an estimated Maximum Torque can be 27 Nmm (single Xmorph). The designs may need around ~20 Xmorph stacked and working in parallel to generate >500 Nmm.

In FIG. 11, the design 1100 can include first end 1102, second end 1104, SMA wires 1108A-B, and cross-members 1106A-B disposed between the first end 1102 and second end 1104. The cross-members 1106A-B can move slightly as torque is generated in response to actuation by the SMA wires 1108A-B. The SMA wires 1108A-B can be actuated in response to a current being applied by a power source, and a controller can selectively control the electrical current applied to the SMA wires.

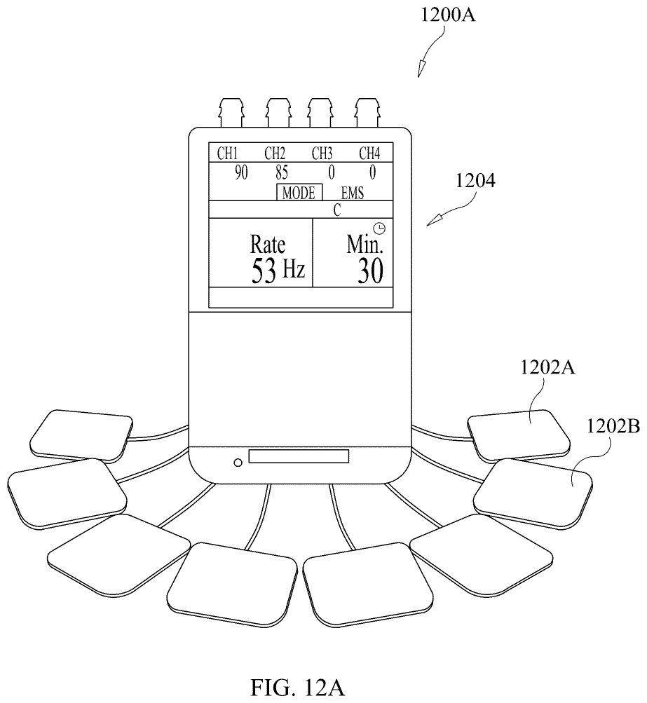

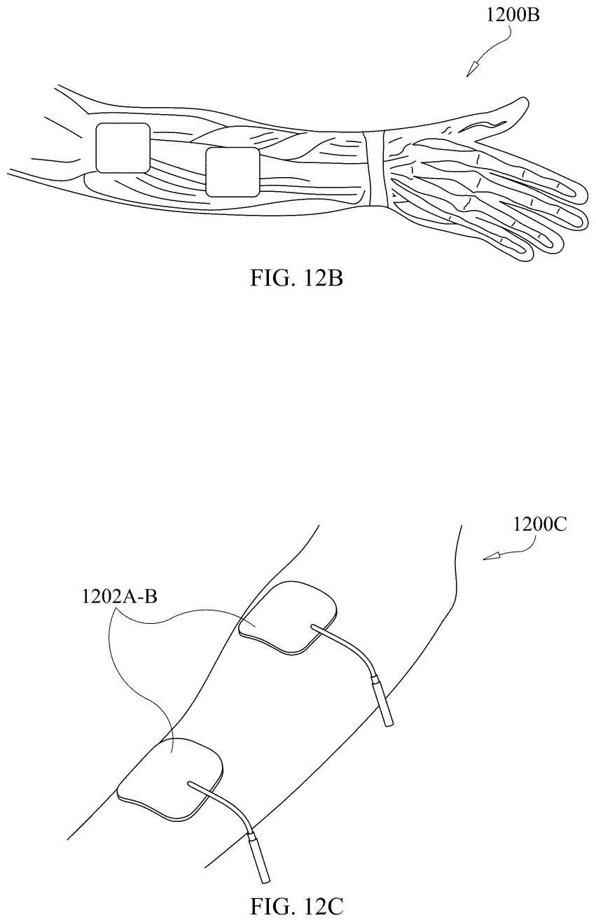

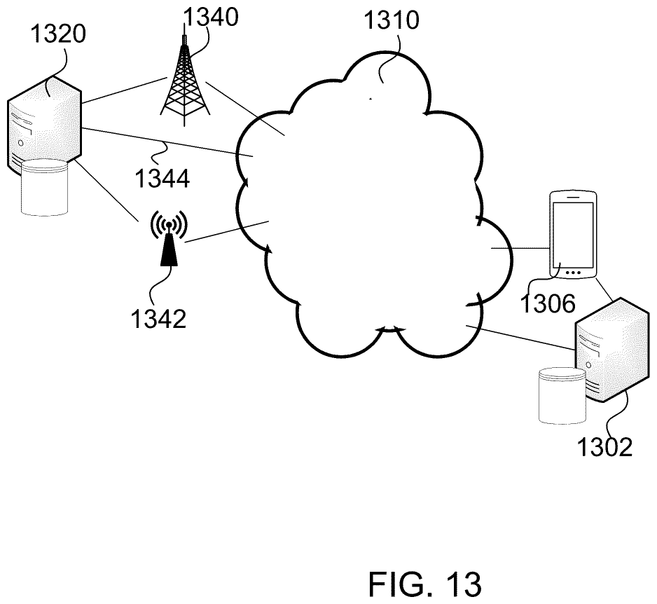

FIGS. 12A-C illustrate views 1200A-C an example EMS system for essential tremor control. As shown in FIG. 12A, a first view of a EMS system 1200A can include a controller 1204 that can control actuation of SMA wires connected different pads 1202A, 1202B. Controller 1204 can include a computing device that can cause a display of specific parameters along with an ability to cause an electrical current to be provided to the SMA wires in each pad 1202A-B.

Each pad 1202A-B can include a SMA wire and can actuate to activate specific muscles in the arm of the user. For instance, in FIG. 12C, pads 1202A-B can be disposed on parts of the arm of the user such that muscles are activated in response to the actuation of the SMA wire at any of the pads 1202A-B.

The designs as shown in FIG. 12A-12C can simulate forearm muscles with electrical pulses driven based on IMU feedback. In an example, four pads (2 top/2 bottom) may be enough to control up and down hand motion.

In some embodiments, the systems as described herein can include SMA actuators that have the force to move the 0.5 kg hand up and down ~±10°. The designs can include any of a straight SMA Wire concept or a stacked Xmorph concept. Analysis can reveal a large force on forearm ≥50N may be needed to anchor the actuator. Adding semi-compliant links between straps can help distribute this force. These forces may cause circulation issues, and flexing of the skin can reduce performance of the actuator.

In some embodiments, SMA wires can function as position sensors in the systems as described herein. In an example, a relatively small and constant current can be applied to SMA wire circuits for a measurement of resistance that can relate to position. A motion control structure can be added to the structures as described herein to use a resistance or a voltage of the SMA wires to determine position of elements of the system, such as the glove, wrist portion, or the forearm portion as described herein.

The resistance of the SMA wires and thereby a position of the elements of the system can be measured over time to determine not only the position of the system but also whether a tremor is present. For instance, the position of the system elements can be tracked via SMA position sensing such that rapid and sudden position changes consistent with a tremor can be detected. In another example, if the hand (e.g., tracked via position of the glove element) is moving slowly relative to the forearm (e.g., tracked via position of the forearm element) without continuous oscillations, then there may not no tremor as the motion can be normal use of the arm of the user. However, if the position of the hand (tracked via position of the glove element) is oscillating relative to the forearm (e.g., tracked via position of the forearm element) continuously or for more than a certain number of cycles, then a tremor can be detected. A controller or computer can process electrical or resistance data from the SMA wires and track the position of the system as described herein.

In an example embodiment, a system for suppressing tremors in a user is provided. The system can include a first component configured to connect to the user. The first component can surround a wrist of the user. In some instances, the first component comprises a set of ball bearing sliding surfaces to allow for sliding movement of the connection pads on the first component.

The system can also include a second component configured to connect to the user. The second component can surround a forearm of the user. In some instances, the first component and the second component comprises a strap with a fastener to secure the strap to the user.

In some instances, the system can also include a set of connection pads on each of the first component and the second component, wherein each of the set of connection pads provides a flat surface to engage the first end or the second end of any of the set of SMA wires to the first component or the second component.

The system can also include a set of shape memory alloy (SMA) wires connecting at a first end to the first component and at a second end to the second component. Actuation of the set of SMA wires can be configured to contract/relax muscles of a user in response to a tremor and allow for movement during normal motion of the user.

In some instances, the set of SMA wires further comprises a first pair of SMA wires on a first side of the first component and the second component and a second pair of SMA wires on a second side of the first component and the second component opposite to the first side.

In some instances, the system can also include a glove component configured to engage with a hand and/or fingers of the user.

In some instances, the system can also include a set of metal wires connecting the glove component to the first component.

In some instances, the system can also include a series of links connecting the first component to the second component and disposed adjacent to any of the set of SMA wires.

In some instances, the system can also include a silicon sleeve disposed around any of the set of SMA wires.

In some instances, the system can also include a set of cross-members disposed between the first component and the second component, wherein the set of cross-members cross at a central point.

In some instances, the system can also include a stack of actuators disposed between the first component and the second component, wherein each actuator in the stack of actuators comprises the set of cross-members and the set of SMA wires.

In some instances, the stack of actuators comprises at least ten actuators in a stacked configuration.

In some instances, the system can also include one or more Electric Muscle Simulation (EMS) devices configured to be disposed on the user, wherein each of the one or more EMS devices comprises a SMA element configured to actuate and cause activation of a muscle of the user.

In some instances, wherein the one or more SMA actuators are configured to generate sufficient force to hold and move the weight of the users'hand and/or wrist while also generating sufficient force to suppress tremors in the user.

In another example, a device is provided. The device can include a glove component configured to connect to a hand and/or fingers of a user. The device can also include a wrist component configured to connect to a wrist region of the user. The device can also include a forearm component configured to connect to a forearm region of the user. The device can also include a set of metal wires disposed between the glove component and the wrist component. The device can also include a set of shape memory alloy (SMA) wires connecting at a first end to the wrist component and at a second end to the forearm component. The set of SMA wires can further include a first pair of SMA wires on a first side of the first component and the second component and a second pair of SMA wires on a second side of the first component and the second component opposite to the first side.

In some instances, the device can also include a set of connection pads on each of the first component and the second component, wherein each of the set of connection pads provides a flat surface to engage the first end or the second end of any of the set of SMA wires to the first component or the second component.

In some instances, the device can also include a series of links connecting the first component to the second component and disposed adjacent to any of the set of SMA wires.

In some instances, the device can also include a set of cross-members disposed between the first component and the second component, wherein the set of cross-members cross at a central point.

In some instances, the device can also include one or more Electric Muscle Simulation (EMS) devices configured to be disposed on the user, wherein each of the one or more EMS devices comprises a SMA element configured to actuate and cause activation of a muscle of the user.

### Network Examples

Systems and methods here may utilize a networked computing arrangement as shown in FIG. 13. The computer used for these steps could be any number of kinds of computers such as those included in a camera itself, and/or another computer arrangement in communication with the camera computer components including but not limited to a mobile device 1306, laptop, desktop, tablet, phablet, smartphone, or any other kind of device used to process and transmit digitized data.

Turning back to FIG. 13, the data captured from whichever computer may be analyzed on a back-end system instead of or in addition to a local computer. In such examples, data may be transmitted to a back-end computer 1320 and associated data storage 1302 for saving, analysis, computation, comparison, or other manipulation. In some examples, additionally or alternatively, the transmission of data may be wireless 1340, 1342 by a cellular or Wi-Fi transmission with associated routers and hubs. In some examples, additionally or alternatively, the transmission may be through a wired connection 1344. In some examples, additionally or alternatively, the transmission may be through a network such as the internet 1310 to the back-end computer 1320 and associated data storage 1302. In some examples, additionally or alternatively, the storing, analyzing, and/or processing of data may be accomplished at the computer which is involved in the original image capture and/or data collection. In some examples, additionally or alternatively, the data storing, analyzing, and/or processing may be shared between the local computer 1306 and a back-end computer 1320. In such examples, networked computer resources may allow for more data processing power to be utilized than may be otherwise available at the local computers. In such a way, the processing and/or storage of data may be offloaded to the compute resources that are available. In some examples, additionally or alternatively, the networked computer resources may be virtual machines in a cloud or distributed infrastructure. In some examples, additionally or alternatively, the networked computer resources may be spread across many multiple physical or virtual computer resources by a cloud infrastructure. The example of a single computer server is not intended to be limiting and is only one example of a compute resource that may be utilized by the systems and methods described herein. In some examples, additionally or alternatively, artificial intelligence and/or machine learning may be used to perform any of the steps as described herein.

### Example Computer Devices

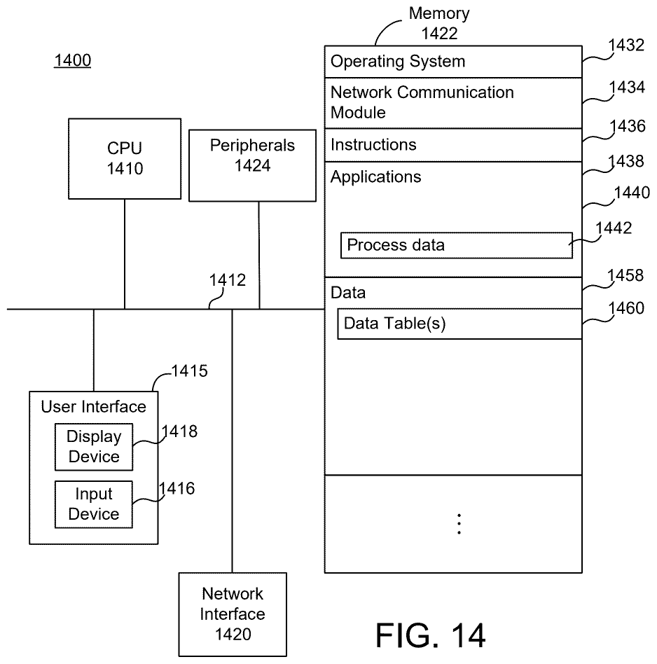

FIG. 14 shows an example computing device 1400 which may be used in the systems and methods described herein. In the example computing device 1400 a CPU or processor 1416 is in communication by a bus or other communication 1412 with a user interface 1415. The user interface includes an example input device such as a keyboard, mouse, touchscreen, button, or other user input device(s). The user interface 1415 also includes a display device 1418 such as a screen. The computing device 1400 shown in FIG. 14 also includes a network interface 1420 which is in communication with the CPU 1410 and other components. The network interface 1420 may allow the computing device 1400 to communicate with other computers, databases, networks, user devices, or any other computing capable devices. In some examples, additionally or alternatively, the method of communication may be through WIFI, cellular, Bluetooth Low Energy, wired communication, or any other kind of communication. In some examples, additionally or alternatively, the example computing device 1400 includes peripherals 1424 also in communication with the processor 1416. In some examples computing device 1400 a memory 1422 is in communication with the processor 1416. In some examples, additionally or alternatively, this memory 1422 may include instructions to execute software such as an operating system 1432, network communications module 1434, other instructions 1436, applications 1438, data storage 1458, data such as data tables 1460 or any other kind of data.

### Conclusion

As disclosed herein, features consistent with the present embodiments may be implemented via computer-hardware, software and/or firmware. For example, the systems and methods disclosed herein may be embodied in various forms including, for example, a data processor, such as a computer that also includes a database, digital electronic circuitry, firmware, software, computer networks, servers, or in combinations of them. Further, while some of the disclosed implementations describe specific hardware components, systems and methods consistent with the innovations herein may be implemented with any combination of hardware, software and/or firmware.

Moreover, the above-noted features and other aspects and principles of the innovations herein may be implemented in various environments. Such environments and related applications may be specially constructed for performing the various routines, processes and/or operations according to the embodiments or they may include a computer or computing platform selectively activated or reconfigured by code to provide the necessary functionality. The processes disclosed herein are not inherently related to any particular computer, network, architecture, environment, or other apparatus, and may be implemented by a suitable combination of hardware, software, and/or firmware. For example, various machines may be used with programs written in accordance with teachings of the embodiments, or it may be more convenient to construct a specialized apparatus or system to perform the methods and techniques described herein.

Aspects of the method and system described herein, such as the logic, may be implemented as functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Some other possibilities for implementing aspects include: memory devices, microcontrollers with memory (such as EEPROM), embedded microprocessors, firmware, software, etc. Furthermore, aspects may be embodied in microprocessors having software-based circuit emulation, discrete logic (sequential and combinatorial), custom devices, fuzzy (neural) logic, quantum devices, and hybrids of any of the above device types. The underlying device technologies may be provided in a variety of component types, e.g., metal-oxide semiconductor field-effect transistor (“MOSFET”) technologies like complementary metal-oxide semiconductor (“CMOS”), bipolar technologies like emitter-coupled logic (“ECL”), polymer technologies (e.g., silicon-conjugated polymer and metal-conjugated polymer-metal structures), mixed analog and digital, and so on.

It should also be noted that the various logic and/or functions disclosed herein may be enabled using any number of combinations of hardware, firmware, and/or as data and/or instructions embodied in various machine-readable or computer-readable media, in terms of their behavioral, register transfer, logic component, and/or other characteristics. Computer-readable media in which such formatted data and/or instructions may be embodied include, but are not limited to, non-volatile storage media in various forms (e.g., optical, magnetic or semiconductor storage media) and carrier waves that may be used to transfer such formatted data and/or instructions through wireless, optical, or wired signaling media or any combination thereof. Examples of transfers of such formatted data and/or instructions by carrier waves include, but are not limited to, transfers (uploads, downloads, e-mail, etc.) over the Internet and/or other computer networks via one or more data transfer protocols (e.g., HTTP, FTP, SMTP, and so on).

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. Additionally, the words “herein,” “hereunder,” “above,” “below,” and words of similar import refer to this application as a whole and not to any particular portions of this application. When the word “or” is used in reference to a list of two or more items, that word covers all of the following interpretations of the word: any of the items in the list, all of the items in the list and any combination of the items in the list.

It will be appreciated that the term “present invention” as used herein should not be construed to mean that only a single invention having a single essential element or group of elements is presented. Similarly, it will also be appreciated that the term “present invention” encompasses a number of separate innovations, which can each be considered separate inventions. Although the present invention has been described in detail with regards to the preferred embodiments and drawings thereof, it should be apparent to those skilled in the art that various adaptations and modifications of embodiments of the present invention may be accomplished without departing from the spirit and the scope of the invention. Accordingly, it is to be understood that the detailed description and the accompanying drawings as set forth hereinabove are not intended to limit the breadth of the present invention, which should be inferred only from the following claims and their appropriately construed legal equivalents.

What is claimed is:

1. A system for suppressing tremors in a user, comprising:
a first component configured to connect to the user;
a second component configured to connect to the user; and
a set of shape memory alloy (SMA) wires connecting at a first end to the first component and at a second end to the second component, wherein actuation of the set of SMA wires are configured to contract or relax muscles of a user in response to a tremor and allow for movement during normal motion of the user.

2. The system of claim 1, wherein the first component is configured to surround a wrist of the user, and wherein the second component is configured to surround a forearm of the user.

3. The system of claim 1, wherein the first component and the second component comprises a strap with a fastener to secure the strap to the user.

4. The system of claim 1, wherein the set of SMA wires further comprises a first pair of SMA wires on a first side of the first component and the second component and a second pair of SMA wires on a second side of the first component and the second component opposite to the first side.

5. The system of claim 1, further comprising:
a set of connection pads on each of the first component and the second component, wherein each of the set of connection pads provides a flat surface to engage the first end or the second end of any of the set of SMA wires to the first component or the second component.

6. The system of claim 5, wherein the first component comprises a set of ball bearing sliding surfaces to allow for sliding movement of the connection pads on the first component.

7. The system of claim 1, further comprising:
a glove component configured to engage with a hand and/or fingers of the user.

8. The system of claim 7, further comprising:
a set of metal wires connecting the glove component to the first component.

9. The system of claim 1, further comprising:
a series of links connecting the first component to the second component and disposed adjacent to any of the set of SMA wires.

10. The system of claim 1, further comprising:
a silicon sleeve disposed around any of the set of SMA wires.

11. The system of claim 1, further comprising:
a set of cross-members disposed between the first component and the second component, wherein the set of cross-members cross at a central point.

12. The system of claim 11, further comprising:
a stack of actuators disposed between the first component and the second component, wherein each actuator in the stack of actuators comprises the set of cross-members and the set of SMA wires.

13. The system of claim 12, wherein the stack of actuators comprises at least ten actuators in a stacked configuration.

14. The system of claim 11, further comprising:
one or more Electric Muscle Simulation (EMS) devices configured to be disposed on the user, wherein each of the one or more EMS devices comprises a SMA element configured to actuate and cause activation of a muscle of the user.

15. The system of claim 1, wherein the set of SMA wires are configured to generate sufficient force to hold and move a weight of a hand of the users and/or wrist while also generating sufficient force to suppress tremors in the user.

16. A device comprising:
a glove component configured to connect to a hand and/or fingers of a user;
a wrist component configured to connect to a wrist region of the user;
a forearm component configured to connect to a forearm region of the user;
a set of metal wires disposed between the glove component and the wrist component; and
a set of shape memory alloy (SMA) wires connecting at a first end to the wrist component and at a second end to the forearm component, wherein the set of SMA wires further comprises a first pair of SMA wires on a first side of the first component and the second component and a second pair of SMA wires on a second side of the first component and the second component opposite to the first side.

17. The device of claim 16, further comprising:
a set of connection pads on each of the first component and the second component, wherein each of the set of connection pads provides a flat surface to engage the first end or the second end of any of the set of SMA wires to the first component or the second component.

18. The device of claim 16, further comprising:
a series of links connecting the first component to the second component and disposed adjacent to any of the set of SMA wires.

19. The device of claim 16, further comprising:
a set of cross-members disposed between the first component and the second component, wherein the set of cross-members cross at a central point.

20. The device of claim 16, further comprising:
one or more Electric Muscle Simulation (EMS) devices configured to be disposed on the user, wherein each of the one or more EMS devices comprises a SMA element configured to actuate.
